(19) United States
(12) Patent Application Publication
(10) Pub. No.: US 2004/0142353 A1
(43) Pub. Date: Jul. 22, 2004

(54) Compositions and methods for identifying plants having increased tolerance to imidazolinone herbicides

(76) Inventors: Wing Y. Cheung, Brossard (CA); Marie-Josee Gagnon, Montreal (CA); Martin Laforest, St-Luc (CA); Benoit S. Landry, L'Acadie (CA)

Correspondence Address: BASF CORPORATION, 26 DAVIS DRIVE, RESEARCH TRIANGLE PARK, NC, 27709, US

(21) Appl. No.: 10/695,089

(22) Filed: Oct. 28, 2003

Related U.S. Application Data
(60) Provisional application No. 60/421,993, filed on Oct. 29, 2002.

(51) Int. Cl. C12Q 1/68; C07H 21/04

(52) U.S. Cl. 435/6; 536/23.2

(57) ABSTRACT

The present invention provides compositions and methods for assaying commercially relevant imidazolinone herbicide tolerance conferred by a Brassica napus AHAS1 PM1 mutation and a Brassica napus AHAS3 PM2 mutation in a plant.

This application claims priority under 35 U.S.C. § 119(e) to U.S. provisional application serial No. 60/421,993, filed Oct. 29, 2002.

This invention relates generally to compositions and methods for identifying Brassica plants having increased tolerance to an imidazolinone herbicide.

### BACKGROUND OF THE INVENTION

Canola is the seed derived from any of the Brassica species B. napus, B. campestris/rapa, and certain varieties of B. juncea. Canola oil is high in monounsaturated fats, moderate in polyunsaturated fats, and low in saturated fats, having the lowest level of saturated fat of any vegetable oil. Thus canola oil is an important dietary option for lowering serum cholesterol in humans. In addition, the protein meal which is the byproduct of canola oil production has a high nutritional content and is used in animal feeds.

Imidazolinone and sulfonylurea herbicides are widely used in modern agriculture due to their effectiveness at very low application rates and relative non-toxicity in animals. Both of these herbicides act by inhibiting acetohydroxyacid synthase (AHAS; EC 4.1.3.18, also known as acetolactate synthase or ALS), the first enzyme in the synthetic pathway of the branched chain amino acids valine, leucine and isoleucine. Several examples of commercially available imidazolinone herbicides are PURSUIT® (imazethapyr), SCEPTER® (imazaquin) and ARSENAL® (imazapyr). Examples of sulfonylurea herbicides are chlorsulfuron, metsulfuron methyl, sulfometuron methyl, chlorimuron ethyl, thifensulfuron methyl, tribenuron methyl, bensulfuron methyl, nicosulfuron, ethametsulfuron methyl, rimsulfuron, triflusulfuron methyl, triasulfuron, primisulfuron methyl, cinosulfuron, amidosulfuron, fluzasulfuron, imazosulfuron, pyrazosulfuron ethyl and halosulfuron.

Due to their high effectiveness and low toxicity, imidazolinone herbicides are favored for application to many crops, including canola, by spraying over the top of a wide area of vegetation. The ability to spray an herbicide over the top of a wide range of vegetation decreases the costs associated with plantation establishment and maintenance and decreases the need for site preparation prior to use of such chemicals. Spraying over the top of a desired tolerant species also results in the ability to achieve maximum yield potential of the desired species due to the absence of competitive species. However, the ability to use such spray-over techniques is dependent upon the presence of imidazolinone resistant species of the desired vegetation in the spray over area. In addition, because residual imidazolinones persist in a sprayed field, a variety of resistant species is advantageous for crop rotation purposes.

Unfortunately, the Brassica species which are the source of canola are closely related to a number of broad leaf cruciferous weeds, for example, stinkweed, ball mustard, wormseed mustard, hare's ear mustard, shepherd's purse, common peppergrass, flixweed, and the like. Thus it was necessary to develop Brassica cultivars which are tolerant or resistant to the imidazolinone herbicides. Swanson, et al. (1989) Theor. Appl. Genet. 78, 525-530 discloses B. napus mutants P₁ and P₂, developed by mutagenesis of microspores of B. napus (cv ‘Topas’), which demonstrated tolerance to the imidazolinone herbicides PURSUIT® and ASSERT® at levels approaching ten times the field-recommended rates. The homozygous P₂ mutant produced an AHAS enzyme which was 500 times more tolerant to PURSUIT® than wild type enzyme, while the AHAS enzyme from the homozygous P₁ mutant was only slightly more tolerant than the wild type enzyme. In field trials, the P₁, P₂, and P₁×P₂ hybrid withstood ASSERT® applications up to 800 g/ha with no loss of yield. The P₁ and P₂ mutations were unlinked and semidominant, and P₁×P₂ crosses tolerated levels of PURSUIT® higher than those tolerated by either homozygous mutant. Imidazolinone-tolerant cultivars of B. napus were developed from the P₁×P₂ mutants and have been sold as CLEARFIELD® canola. See also, Canadian patent application number 2,340,282; Canadian patent number 1,335,412, and European patent number 284419.

Rutledge, et al. (1991) Mol. Gen. Genet. 229, 31-40) discloses the nucleic acid sequence of three of the five genes encoding AHAS isoenzymes in B. napus, AHAS1, AHAS2, and AHAS3. Rutledge, et al. discusses the mutants of Swanson, et al. and predicts that the two alleles that conferred resistance to imidazolinone herbicides correspond to AHAS1 and AHAS3. Hattori et al. (1995) Mol. Gen. Genet. 246, 419-425 disclose a mutant allele of AHAS3 from a mutant B. napus cv Topas cell suspension culture line in which a single nucleotide change at codon 557 leading to an amino acid change from tryptophan to leucine confers resistance to sulfonylurea, imidazolinone, and triazolopyrimidine herbicides. Codon 557 of Hattori, et al. corresponds to codon 556 of the AHAS3 sequence disclosed in Rutledge, et al., supra, and to codon 556 of the AHAS3 sequence set forth as GENBANK accession number gi/17775/emb/Z 11526/.

A single nucleotide mutation at codon 173 in a B. napus ALS gene, corresponding to AHAS2 of Rutledge et al., supra, leads to a change from Pro to Ser (Wiersma et al. (1989) Mol. Gen. Genet. 219, 413-420). The mutant B. napus AHAS2 gene was transformed into tobacco to produce a chlorsulfuron tolerant phenotype.

U.S. Pat. Nos. 6,114,116 and 6,358,686 disclose nucleic acid sequences from B. napus and B. oleracea containing polymorphisms, none of which appears to correspond to the polymorphism disclosed in Hattori, et al., supra.

For commercially relevant Brassica cultivars, it is necessary to ensure that each lot of herbicide-resistant seed contains all mutations necessary to confer herbicide tolerance. A method is needed to detect mutations in Brassica AHAS1 and AHAS3 genes that confer increased imidazolinone tolerance to commercial cultivars.

### SUMMARY OF THE INVENTION

The present invention describes the location and identity of a single nucleotide polymorphism at position 1937 of the AHAS1 gene of B. napus, the polymorphism being designated as the PM1 mutation. The PM1 mutation confers about 15% of the tolerance to imidazolinone herbicides that is present in CLEARFIELD® canola. CLEARFIELD® canola also contains a second single nucleotide polymorphism at position 1709 of the AHAS3 gene of B. napus, which corresponds to the tryptophan to leucine substitution described in Hattori et al., supra. For the purpose of the present invention, this polymorphism is designated as the PM2 mutation. The PM2 mutation confers about 85% of the tolerance to imidazolinone herbicides exhibited by CLEARFIELD® canola. Both the PM1 and PM2 mutations are required to produce a Brassica plant with sufficient herbicide tolerance to be commercially relevant, as in CLEARFIELD® canola.

Accordingly, the present invention provides methods of identifying a plant having increased tolerance to an imidazolinone herbicide by detecting the presence or absence of the B. napus PM1 and PM2 mutations in the plant. One of the advantages of the present invention is that it provides a reliable and quick means to detect plants with commercially relevant imidazolinone tolerance.

In one embodiment, the invention provides a method of assaying a plant for imidazolinone herbicide resistance conferred by the combination of the PM1 mutation of the B. napus AHAS1 gene and the PM2 mutation of the B. napus AHAS3 gene. In this method, genomic DNA is isolated from the plant, the presence or absence of the PM1 mutation is determined, and the presence or absence of the PM2 mutation is determined, wherein the presence of the PM1 mutation and the PM2 mutation is indicative of commercially relevant imidazolinone tolerance in the plant.

In another embodiment, the invention provides novel polynucleotide primers useful for detecting the PM1 and PM2 mutations.

### BRIEF DESCRIPTION OF THE DRAWINGS

FIG. 1A shows the nucleic acid and amino acid sequences of B. napus AHAS1 containing the PM1 mutation (SEQ ID NO:1 and SEQ ID NO:101, respectively).

FIG. 1B shows the nucleic acid and amino acid sequences of B. napus AHAS3 containing the PM2 mutation (SEQ ID NO:2 and SEQ ID NO:102, respectively).

FIG. 1C shows the nucleic acid and amino acid sequences of wild type B. napus cv. ‘Topas’ AHAS1 (SEQ ID NO:3 and SEQ ID NO:103, respectively).

FIG. 1D shows the nucleic acid and amino acid sequences of wild type B. napus AHAS3 Topas cv. (SEQ ID NO:4 and SEQ ID NO:104, respectively).

FIG. 1E is a table setting forth the sequences of various oligonucleotides (SEQ ID NOs: 5-88) useful in determining the presence or absence of the PM1 and PM2 mutations in accordance with the invention.

FIG. 2 is a schematic representation of one embodiment of the PM1 mutation determination step of a primer extension-based assay of the invention. The coding strand is shown with the amino acid translation of the codons. The wild type plant is denoted as ‘Topas’ (SEQ ID NOs:105, 106, 24, 105, 106, and 107, respectively, in order of appearance) and the mutated plant is denoted as ‘PM1’ (SEQ ID NOs:108, 109. 24, 108, 109, and 110, respectively, in order of appearance). The mutated nucleotide “A” is underlined on the coding strand. The PM1 extension primer is indicated in bold and is placed at its annealing site on AHAS1.

FIG. 3 is a schematic representation of one embodiment of the PM2 mutation determination step of a primer extension-based assay of the invention. The coding strand is shown with the amino acid translation of the codons. The wild type plant is denoted as ‘Topas’ (Seq ID NOs:111, 112, 66, 111, 112, and 113, respectively, in order of appearance) and the mutated plant is denoted as ‘PM2’ (SEQ ID NOs:114, 115, 66, 114, 115, and 116, respectively, in order of appearance). The mutated nucleotide “T” is underlined on the coding strand. The PM2 extension primer is indicated in bold and is placed at its annealing site on AHAS3.

FIG. 4 is a table describing the predicted phenotypes of double haploid B. napus plants used to validate the method of the invention.

FIG. 5 is a table describing the results of the method of the invention in an embodiment employing the ABI PRISM® SNP detection system.

FIG. 6 is a table describing the results of the method of the invention in an embodiment employing the PYROSEQUENCING™ detection system.

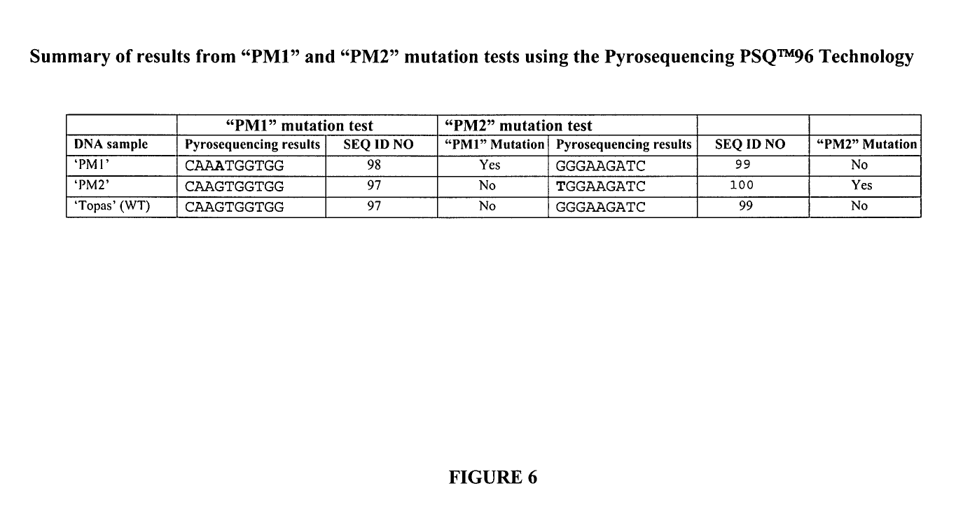

### DETAILED DESCRIPTION OF THE INVENTION

The present invention provides methods and compositions for identifying plants having increased tolerance to an imidazolinone herbicide by virtue of the presence of the B. napus PM1 and PM2 mutations. More particularly, the methods and compositions of the present invention allow identification of Brassica seeds and plants having commercially relevant imidazolinone tolerance, such as CLEARFIELD® canola. In some embodiments, the methods of the invention employ novel polynucleotide primers including PM1 extension primers and PM2 extension primers.

It is to be understood that as used in the specification and in the claims, “a” or “an” can mean one or more, depending upon the context in which it is used. Thus, for example, reference to “a cell” can mean that at least one cell can be utilized.

For the purposes of the present invention, the level of tolerance to imidazolinone herbicides exhibited by CLEARFIELD® canola which contains both the PM1 and PM2 mutations is defined as 100% tolerance, or “commercially relevant imidazolinone tolerance” or “commercial field tolerance”. The terms “tolerance” and “resistance” are used interchangeably herein.

“Homologs” are defined herein as two nucleic acids or polypeptides that have similar, or “identical”, nucleotide or amino acid sequences, respectively. Homologs include allelic variants, analogs, orthologs and paralogs. As used herein, the term “allelic variant” refers to a nucleotide sequence containing polymorphisms that lead to changes in the amino acid sequences of AHAS proteins and that exist within a natural population (e.g., a plant species or variety). As used herein, the term “analogs” refers to two nucleic acids that have the same or similar function, but that have evolved separately in unrelated organisms. The term “orthologs” refers to two nucleic acids from different species, but that have evolved from a common ancestral gene by speciation. Normally, orthologs encode polypeptides having the same or similar functions. As also used herein, the term “paralogs” refers to two nucleic acids that are related by duplication within a genome. Paralogs usually have different functions, but these functions may be related (Tatusov, R. L. et al., 1997 Science 278(5338):631-637).

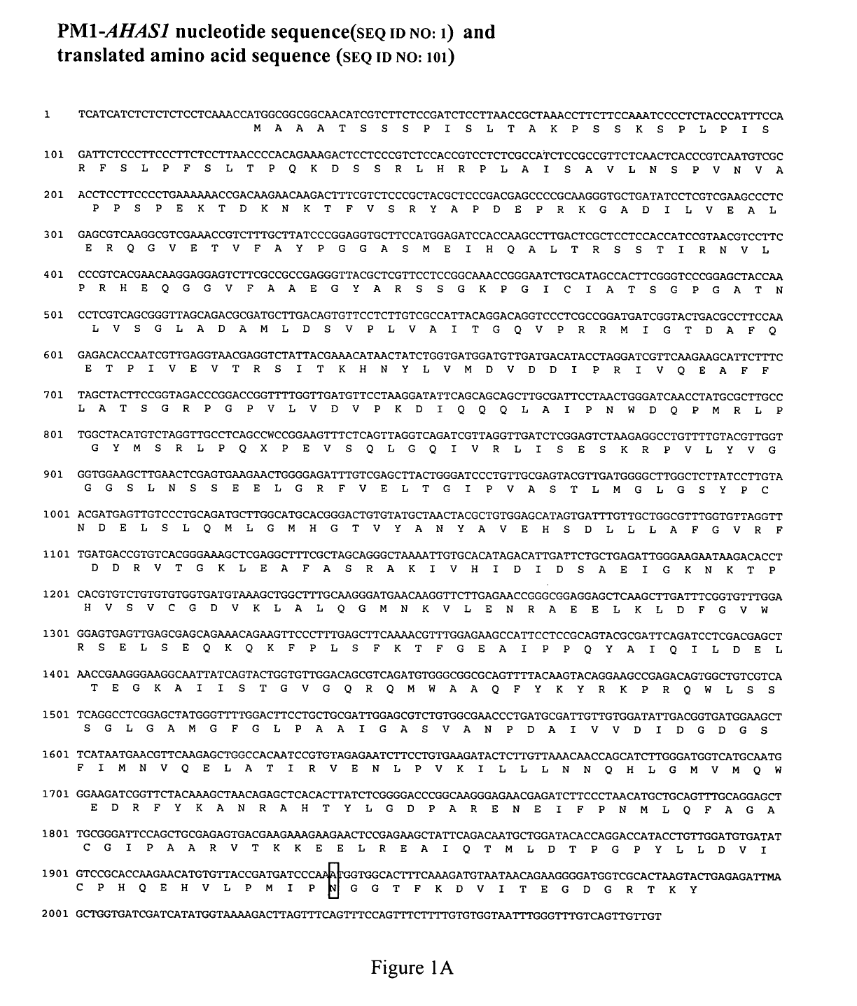

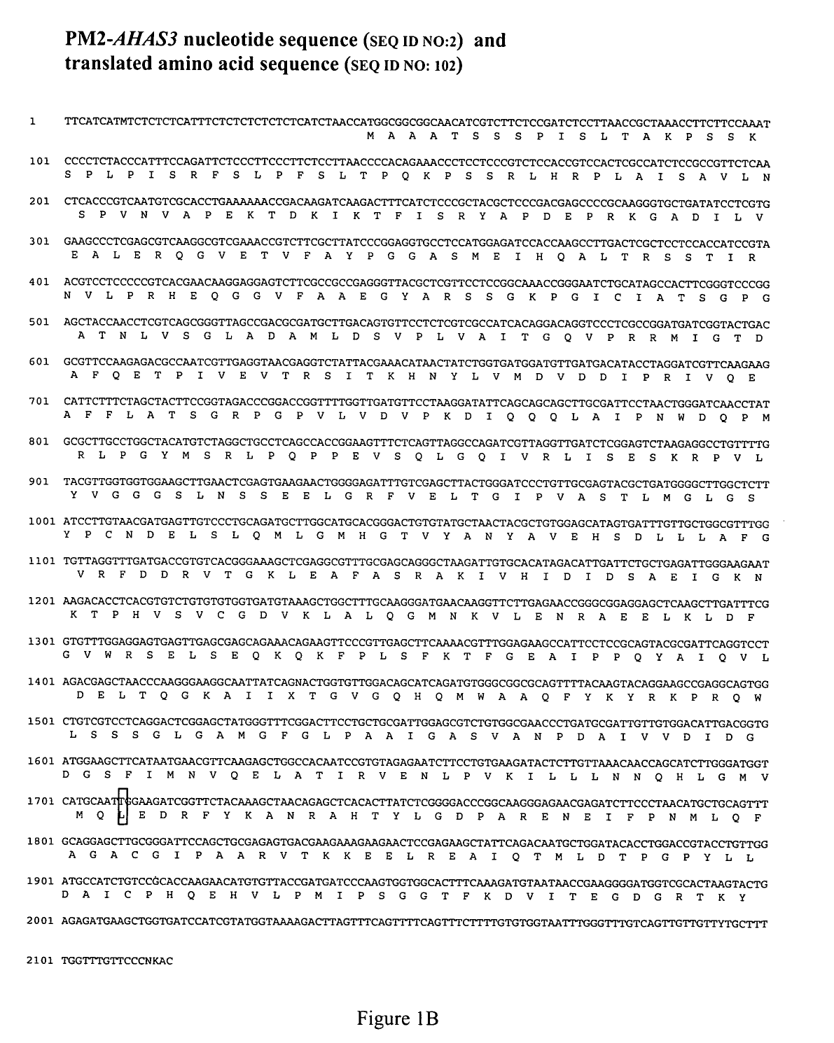

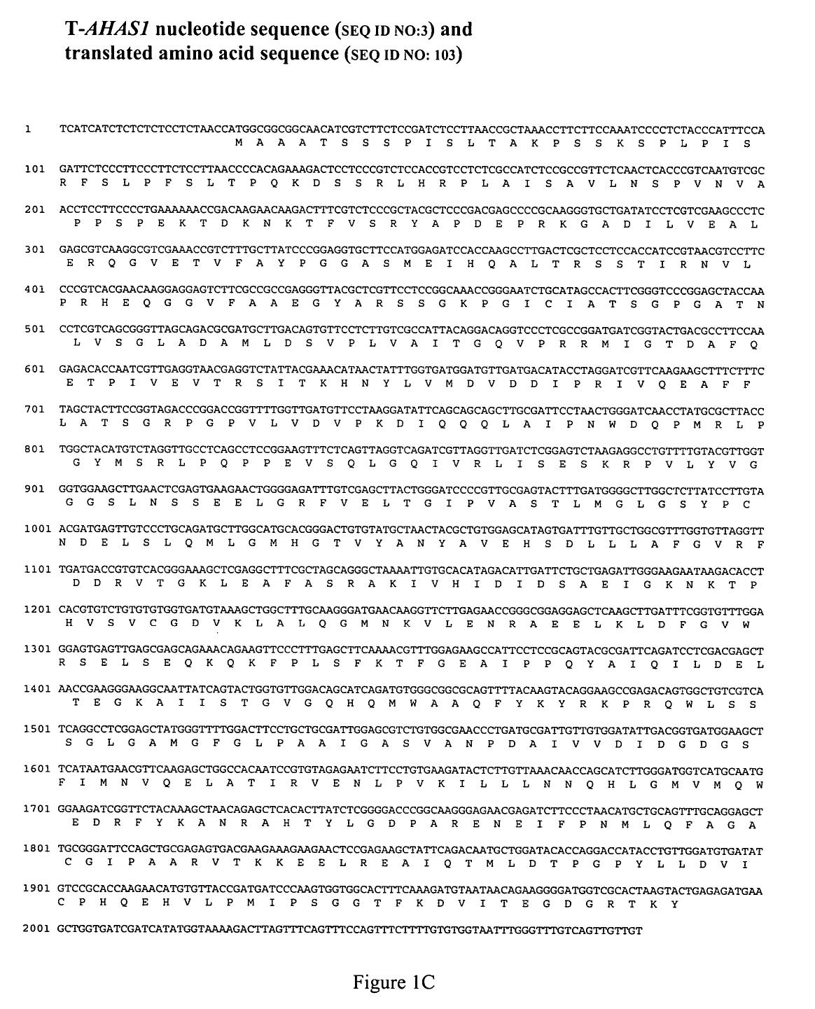

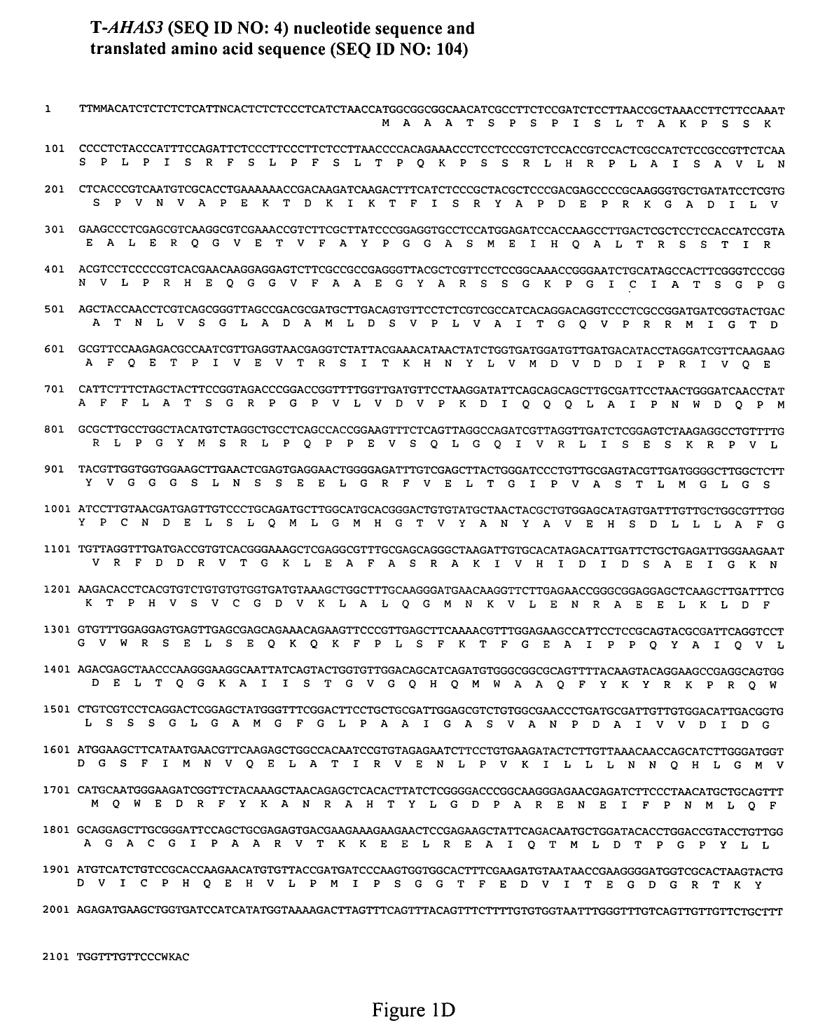

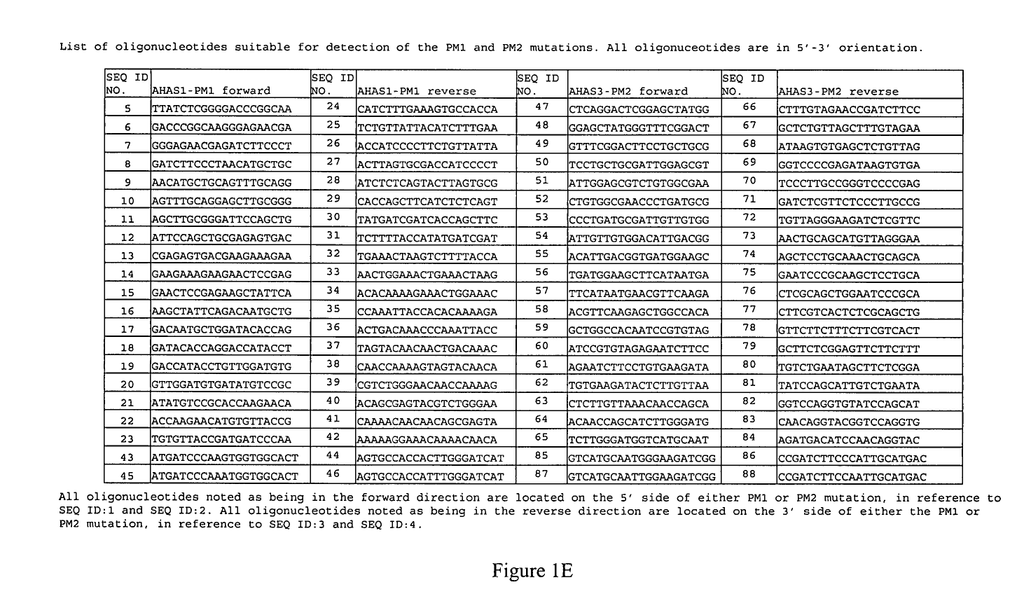

As defined herein, a “PM1 mutation” refers to a single nucleotide polymorphism in a B. napus AHAS1 gene in which there is a “G” to “A” nucleotide substitution at position 1937 of the AHAS1 wildtype polynucleotide sequence shown in FIG. 1C (SEQ ID NO:3) or at a nucleotide position that corresponds to position 1937 in an AHAS1 homolog, which substitution leads to a serine to asparagine amino acid substitution at position 638 in the B. napus AHAS1 enzyme.

A “PM1 oligonucleotide” refers to an oligonucleotide sequence corresponding to a PM1 mutation. An oligonucleotide as defined herein is a nucleic acid comprising from about 8 to about 25 covalently linked nucleotides. In accordance with the invention, an oligonucleotide may comprise any nucleic acid, including, without limitation, phosphorothioates, phosphoramidates, peptide nucleic acids, and the like. As defined herein, “corresponding to a PM1 mutation” includes the following: an oligonucleotide capable of specific hybridization to a region of an AHAS1 gene which is 5′ of position 1937 of the AHAS1 gene as set forth in SEQ ID NO:3 (for example, an oligonucleotide comprising any one of SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; or SEQ ID NO:23 as set forth in FIG. 1E); an oligonucleotide capable of specific hybridization to a region of an AHAS1 gene which is 3′ of position 1937 of the AHAS1 gene as set forth in SEQ ID NO:3 (for example, an oligonucleotide comprising any one of SEQ ID NO:24; SEQ ID NO:25; SEQ ID NO:26; SEQ ID NO:27; SEQ ID NO: 28; SEQ ID NO:29; SEQ ID No;30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; or SEQ ID NO:42 as set forth in FIG. 1E); an oligonucleotide capable of specific hybridization to a region of the AHAS1 gene which spans position 1937 of the AHAS1 gene as set forth in SEQ ID NO:3 (for example, an oligonucleotide comprising SEQ ID NO: 45 as set forth in FIG. 1E); an oligonucleotide capable of specific hybridization to a region of an AHAS1 gene which is 5′ of position 1937 of the complement of the AHAS1 gene set forth in SEQ ID NO:3; an oligonucleotide capable of specific hybridization to a region of an AHAS1 gene which is 3′ of position 1937 of the complement of the AHAS1 gene set forth in SEQ ID NO:3; and an oligonucleotide capable of specific hybridization to a region of the AHAS1 gene which spans position 1937 of the complement of the AHAS1 gene as set forth in SEQ ID NO:3 (for example, an oligonucleotide comprising SEQ ID NO: 46 as set forth in FIG. 1E). The term “nucleic acid” includes RNA or DNA that is linear or branched, single or double stranded, or a hybrid thereof. These terms also encompass RNA/DNA hybrids.

As defined herein, a “PM2 mutation” refers to a single nucleotide polymorphism in a B. napus AHAS3 gene in which there is a “G” to “T” nucleotide substitution at position 1709 of the AHAS3 wildtype polynucleotide sequence shown in FIG. 1D (SEQ ID NO:4) or at a nucleotide position that corresponds to position 1709 in an AHAS3 homolog, which substitution leads to a tryptophan to leucine amino acid substitution at position 556 in the B. napus AHAS3 enzyme.

A “PM2 oligonucleotide” refers to an oligonucleotide sequence corresponding to a PM2 mutation. As defined herein, “corresponding to a PM2 mutation” includes the following: an oligonucleotide capable of specific hybridization to a region of an AHAS3 gene which is 5′ of position 1709 of the AHAS3 gene as set forth in SEQ ID NO:4 (for example, an oligonucleotide comprising any one of SEQ ID NO:47; SEQ ID NO:48; SEQ ID NO:49; SEQ ID NO:50; SEQ ID NO:51; SEQ ID NO:52; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; or SEQ ID NO:65 as set forth in FIG. 1E); an oligonucleotide capable of specific hybridization to a region of an AHAS3 gene which is 3′ of position 1709 of the AHAS3 gene as set forth in SEQ ID NO:4 (for example, an oligonucleotide comprising any one of SEQ ID NO:66; SEQ ID NO:67; SEQ ID NO:68; SEQ ID NO:69; SEQ ID NO:70; SEQ ID NO:71; SEQ ID NO:72; SEQ ID NO:73; SEQ ID NO:74; SEQ ID NO:75; SEQ ID NO:76; SEQ ID NO:77; SEQ ID NO:78; SEQ ID NO:79; SEQ ID NO:80; SEQ ID NO:81; SEQ ID NO:82; SEQ ID NO:83; and SEQ ID NO:84 as set forth in FIG. 1E); an oligonucleotide capable of specific hybridization to a region of the AHAS3 gene which spans position 1709 of the AHAS3 gene as set forth in SEQ ID NO:4 (for example, an oligonucleotide comprising SEQ ID NO: 85 as set forth in FIG. 1E); an oligonucleotide capable of specific hybridization to a region of an AHAS3 gene which is 5′ of position 1709 of the complement of the AHAS3 gene set forth in SEQ ID NO:4; an oligonucleotide capable of specific hybridization to a region of an AHAS3 gene which is 3′ of position 1709 of the complement of the AHAS3 gene set forth in SEQ ID NO:4; and an oligonucleotide capable of specific hybridization to a region of the AHAS3 gene which spans position 1709 of the complement of the AHAS3 gene as set forth in SEQ ID NO:4 (for example, an oligonucleotide comprising SEQ ID NO: 86 as set forth in FIG. 1E).

Also encompassed in the present invention are oligonucleotides corresponding to the wild type alleles at the PM1 and PM2 mutations which are useful as controls in the SNP detection assays. For example, an oligonucleotide corresponding to position 1937 of the AHAS1 gene set forth in SEQ ID NO:1, comprising a sequence selected from the group consisting of SEQ ID NO:43 and SEQ ID NO:44 as set forth in FIG. 1E, is useful as a control in a SNP assay for the PM1 mutation. Similarly, an oligonucleotide corresponding to position 1709 of the AHAS3 gene set forth in SEQ ID NO:2, comprising a sequence selected from the group consisting of SEQ ID NO:85 and SEQ ID NO:86 as set forth in FIG. 1E, is useful as a control in a SNP assay for the PM2 mutation.

The presence of the PM1 and PM2 mutations in a plant may confer tolerance to such imidazolinone herbicides as PURSUIT® (imazethapyr, 2-[4,5-dihydro-4-methyl-4-(1-methylethyl)-5-oxo-1H-imidazol-2-yl]-5-ethyl-3-pyridinecarboxylic acid), CADRE® (imazapic, 2-[4,5-dihydro-4-methyl-4-(1-methylethyl)-5-oxo-1H-imidazol-2-yl]-5-methyl-3-pyridinecarboxylic acid), RAPTOR® (imazamox, 2-[4,5-dihydro-4-methyl-4-(1-methylethyl)-5-oxo-1H-imidazol-2-yl]-5-(methoxymethyl)-3-pyridinecarboxylic acid), SCEPTER® (imazaquin, 2-(4,5-dihydro-4-methyl-4-(1-methylethyl)-5-oxo-1H-imidazol-2-yl)-3-quinolinecarboxylic acid), ASSERT® (imazethabenz, methyl esters of 2-[4,5-dihydro-4-methyl-4-(1-methylethyl)-5-oxo-1H-imidazol-2-yl]-4-methylbenzoic acid and 2-[4,5-dihydro-4-methyl-4-(1-methylethyl)-5-oxo-1H-imidazol-2-yl]-5-methylbenzoic acid), ARSENAL® (imazapyr, 2-[4,5-dihydro-4-methyl-4-(1-methylethyl)-5-oxo-1H-imidazol-2-yl]-3-pyridinecarboxylic acid), and the like. In addition, the PM1 and PM2 mutations may confer resistance to sulfonylurea and triazolopyrimidine herbicides.

The PM1 and PM2 mutations may be present in a plant by virtue of mutagenesis of any species of plant containing the B. napus AHAS1 and AHAS3 genes, respectively. Alternatively, the PM1 and PM2 mutations may be present in a plant by virtue of transformation of the B. napus AHAS1 PM1 gene and the B. napus AHAS3 PM2 genes into the plant, using known methods such as those set forth in U.S. Pat. Nos. 5,591,616; 5,767,368; 5,736,369; 6,020,539; 6,153,813; 5,036,006; 5,120,657; 5,969,213; 6,288,312; 6,258,999, and the like. Preferably, the plant is a Brassica oilseed. More preferably, the plant species is selected from the group consisting of B. napus, B. campestris/rapa, and B. juncea. Most preferably, the plant species is B. napus. In accordance with the present invention, the term “plant” includes seeds, leaves, stems, whole plants, organelles, cells, and tissues.

In the first step of the method of the invention, genomic DNA is isolated from the plant. It is to be understood that when practicing the method of the present invention, genomic DNA can be extracted from the plant by any method known to those of skill in the art. Genomic DNA can be extracted from a whole plant, a plant leaf, a plant stem, a plant seed, or any plant organelle, cell or tissue. One non-limiting method for extracting the DNA from a plant leaf is described in Example 1 below.

In the second step of the method of the invention, the presence or absence of the PM1 mutation in the extracted DNA is determined. In the third step of the invention, the presence or absence of the PM2 mutation in the extracted DNA is determined. In accordance with the invention, the steps of detecting the PM1 and PM2 mutations may be performed in any order, or simultaneously.

Any method may be used to detect the PM1 and PM2 mutations. For example, commercially available single nucleotide polymorphism (SNP) detection systems may be used, such as the SNP-IT™ system (Orchid Biosciences, Princeton, N.J.), the MassArray™ System (Sequenom, Inc., San Diego, Calif.), the BeadArray™ System (Illumina, San Diego, Calif.), the ABIPrism Genetic Analyzer (Applied Biosystems, Foster City, Calif.), the ALFexpress™ (Amersham Biosciences, Buckinghamshire, UK), the PSQ™96 System (Pyrosequencing AB, Uppsala, Sweden), the lnvader™ assay (Third Wave Agbio, Inc., Madison, Wis.), and the like. A variety of methods exist for identification of a nucleotide at a polymorphic site in a nucleic acid, as described, for example, in U.S. Pat. Nos. 6,087,095; 6,046,005; 6,017,702; 5,981,186; 5,976,802; 5,928,906; 5,912,118; 5,908,755; 5,869,242; 5,853,979; 5,849,542; 5,834,189; 4,851,331; 4,656,127; 5,679,524; 6,004,744; 6,013,431; 6,210,891; 6,183,958; 5,958,692; 5,851,770; 6,110,684; 5,856,092; 5,605,798; 5,547,835; 6,194,144; 6,043,031; 6,322,980; 6,340,566, and the like. Such technologies include, but are not limited to, allele-specific primer extension, allele-specific hybridization, allele-specific ligation, allele-specific enzymatic cleavage, mismatch detection using resolvase, and sequencing. These technologies can be combined with different signal detection technologies such as fluorescence, fluorescence resonance energy transfer, fluorescence polarization, luminescence and mass spectroscopy.

In some embodiments of the method of the invention, the isolated DNA is combined with a PM1 extension primer and a PM2 extension primer, as defined below, in the presence of one or more SNP detection reagents, thereby creating a detection product. The detection product is then examined to determine the presence or absence of a PM1 mutation or a PM2 mutation in the isolated DNA. As used herein, the term “SNP detection reagent” refers to any reagent that is part of any SNP technology, technique or kit that can be used to detect single nucleotide polymorphisms.

In one embodiment, the template DNA is combined with a first extension primer which is suitable for detection of a PM1 mutation, a second extension primer suitable for detection of a PM2 mutation, and one or more SNP detection reagents. An “extension primer” is an oligonucleotide that binds to the target DNA upstream from the target mutation in the direction of extension. In accordance with the invention, a PM1 extension primer comprises an oligonucleotide corresponding to a PM1 mutation. Similarly, a PM2 extension primer comprises an oligonucleotide corresponding to a PM2 mutation. The extension primer will preferably have a length from about 12 nucleotides to about 100 nucleotides, and more preferably have a length from about 18 nucleotides to about 60 nucleotides.

The extension primer may be chosen to bind substantially uniquely to a target sequence containing a PM1 or PM2 mutation under the conditions of primer extension, so that the sequence will normally be one that is conserved or the primer is long enough to bind in the presence of a few mismatches, usually fewer than about 10% mismatches. By knowing the sequence that is upstream from the PM1 or PM2 mutation, one can select a sequence that has a high G-C ratio, so as to have a high binding affinity for the target sequence. In addition, the extension primer should bind reasonably close to the PM1 or PM2 mutation, preferably not more than about 200 nucleotides away, more preferably not more than about 100 nucleotide away, and most preferably within 50 nucleotides. In a preferred embodiment, the extension primer binds between 1 and 5 nucleotides away from the PM1 or PM2 mutation.

Both the PM1 extension primer and the PM2 extension primer described herein are preferred extension primers. In one embodiment of the present invention, the PM1 extension primer comprises a sequence as shown in SEQ ID NO:24, or any contiguous primer, noncontiguous primer or homologous primer thereof. In another or further embodiment of the present invention, the PM2 extension primer comprises a sequence as shown in SEQ ID NO:66, or any contiguous primer, noncontiguous primer or homologous primer thereof. The PM1 or PM2 primer can also comprise an RNA version of any of the aforementioned extension primers.

The term “contiguous primer” refers to a polynucleotide sequence that contains at least a fragment of the polynucleotide sequence of SEQ ID NO:24, SEQ ID NO:66, SEQ ID NO:23 or SEQ ID NO:65. In one embodiment, the contiguous primer contains a 5′ or 3′ fragment of SEQ ID NO:24, SEQ ID NO:66, SEQ ID NO:23 or SEQ ID NO:65 in addition to one or more nucleotides complementary to upstream or downstream PM1 or PM2 polynucleotide sequences. For example, a contiguous primer of the PM1 primer shown in SEQ ID NO:24 could comprise a nucleotide sequence of TAC ATCTTTGAAAGTGCCA (SEQ ID NO:89). The term “noncontiguous primer” refers to a sequence that is not contiguous with a PM1 or PM2 primer (i.e., a contiguous fragment of the PM1 or PM2 primer), but which sequence contains portions of a PM1 or PM2 primer sequence sufficient to provide the amplification or detection results obtained with SEQ ID NO:24, SEQ ID NO:66, SEQ ID NO:23 or SEQ ID NO:65. For example, with reference to FIG. 1E, oligonucleotides having SEQ ID NOs: 5-21 are noncontiguous with the PM1 primer having SEQ ID NO:23. Finally, the term “homologous primer” refers to a polynucleotide sequence that is substantially homologous with SEQ ID NO:24, SEQ ID NO:66, SEQ ID NO:23 or SEQ ID NO:65 or a contiguous primer thereof. In a preferred embodiment, the contiguous, non-contiguous or homologous primer has the attributes of an extension primer as described above, and more preferably, binds immediately upstream or downstream from a PM1 or PM2 mutation.

Substantially homologous primers included in the present invention are those that provide detection results in ranges similar to those obtained with the oligonucleotide sequence shown in SEQ ID NO:24, SEQ ID NO:66, SEQ ID NO:23 or SEQ ID NO:65. In a preferred embodiment, a primer substantially homologous to SEQ ID NO:24, SEQ ID NO:66, SEQ ID NO:23 or SEQ ID NO:65 is at least about 50-60%, preferably at least about 60-70%, and more preferably at least about 70-75%, 75-80%, 80-85%, 85-90% or 90-95%, and most preferably at least about 96%, 97%, 98%, 99% or more identical to an entire oligonucleotide sequence shown in SEQ ID NO:24, SEQ ID NO:66, SEQ ID NO:23 or SEQ ID NO:65.

To determine the percent sequence identity of two polynucleotide sequences, the sequences are aligned for optimal comparison purposes (e.g., gaps can be introduced in the sequence of one polynucleotide for optimal alignment with the other polynucleotide). The polynucleotides at corresponding positions are then compared. When a position in one sequence (e.g., a sequence of SEQ ID NO:24, SEQ ID NO:66, SEQ ID NO:23 or SEQ ID NO:65) is occupied by the same nucleotide as the corresponding position in the other sequence, then the molecules are identical at that position. Accordingly, the percent sequence identity between the two sequences is a function of the number of identical positions shared by the sequences (i.e., percent sequence identity=numbers of identical positions/total numbers of positions×100). For the purposes of the invention, the percent sequence identity between two nucleic acid or polypeptide sequences is determined using the Vector NTI 6.0 (PC) software package (InforMax, 7600 Wisconsin Ave., Bethesda, Md. 20814). A gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. It is to be understood that for the purposes of determining sequence identity when comparing a DNA sequence to an RNA sequence, a thymidine nucleotide is equivalent to a uracil nucleotide.

The methods described in the examples employ the coding sequences of the PM1 and PM2 mutations as templates, but the method works equally well with SNP detection assays using the non-coding sequence and the primers. For example, a PM1 extension primer with the non-coding strand as template (^{5′}TGTGTTACCGATGATCCCAA^{3′}; SEQ ID NO:23) and a PM2 extension primer with a non-coding strand as template (^{5′}TCTTGGGATGGTCATGCAAT^{3′}; SEQ ID NO:65) may be used with the ABIPrism SnaPshot assay available from Applied Biosystems (Foster City, Calif.).

Prior to the detection steps, template DNA containing the PM1 and PM2 mutations may optionally be amplified using known methods. Amplification and creation of a DNA template can be achieved using any method known to those of skill in the art including PCR. The term “PCR” as used herein refers to the polymerase chain reaction method of DNA amplification. As will be understood by one of ordinary skill in the art, this term also includes any and all other methods known in the art for nucleic acid amplification requiring an amplification target, at least one primer and a polymerase.

For example, either PM1 template DNA or PM2 template DNA may be amplified by combining the isolated genomic DNA with an appropriate primer set for the amplification of a polynucleotide sequence containing a PM1 or PM2 mutation. Each primer set consists of a forward primer and a reverse primer, each of which can be referred to as an “amplification primer.” In one embodiment of the present invention, AHAS1 and AHAS3 template DNAs may be amplified using a single primer set wherein a first amplification primer comprises the sequence 5′ GGC GTT TGG TGT TAG GTT TGA 3′ (SEQ ID NO:90) and a second amplification primer comprises the sequence 5′CGT CTG GGA ACA ACC AAA AGT 3′ (SEQ ID NO:91). Alternatively, an AHAS1 template DNA may be separately amplified using an AHAS1-specific forward primer 5′ GGA AAG CTC GAG GCT TTC GCT 3′ (SEQ ID NO: 92) and an AHAS1/AHAS3 reverse primer 5′ ATC ACC AGC TTC ATC TCT CAG T 3′ (SEQ ID NO: 93). In this embodiment, an AHAS3 template DNA may be separately amplified using an AHAS3-specific forward primer (5′ GGA AAG CTC GAG GCG TTT GCG 3′; SEQ ID NO: 94) and the AHAS1/AHAS3 reverse primer (5′ ATC ACC AGC TTC ATC TCT CAG T 3′; SEQ ID NO: 93).

Those of ordinary skill will recognize that additional amplification primers may be prepared which are contiguous, noncontiguous or homologous primer to the amplification primers et forth above. The forward and reverse primers can also be an RNA version of any of the aforementioned amplification primers.

The invention is further illustrated by the following examples, which are not to be construed in any way as imposing limitations upon the scope thereof.

### EXAMPLES

### Example 1

Isolation of Genomic DNA from a Plant

The DNA extraction procedure described below (Cheung et al., 1993 PCR Methods and Applications 3:69-70) can be used for both fresh and lyophilized leaf tissues. If fresh leaf tissues are used, the Phenol and chloroform/isoamyl-alcohol extraction steps can be omitted.

Two 5 mm diameter leaf discs made with a paper punch or the equivalent were taken from each leaf sample and immediately placed in 320 μl of sterile extraction buffer containing 200 mM Tris-HCl (pH 8.0), 70 mM EDTA, 2 M NaCl and 20 mM sodium metabisulfite. Leaves were then ground until no visible pieces of tissue remained. Cells were lysed with addition of 80 μl of 5% sodium sarcosyl to each tube and were incubated at 60° C. for an hour. After 15 minutes of centrifugation at 13,800 RPM, the supernatant was transferred to a fresh tube and an equal volume of buffer saturated phenol was added. The contents in the tubes were mixed by inverting a few times and were spun at 13,800 RPM for 5 minutes.

The aqueous phase was then transferred into a fresh tube and an equal volume of chloroform/isoamyl alcohol (24:1 v/v) was added and mixed by inverting tubes a few times and then was spun at 13,800 RPM for 5 minutes. After transferring the aqueous phase to a fresh tube, 180 μl of filter-sterilized 10 M ammonium acetate and 400 μl of isopropanol were then added and left at room temperature for 15 minutes for DNA precipitation. After centrifuging for 15 minutes at 13,800 RPM, the supernatant was removed the pellets were rinsed once in 70% EtOH and left to air dry. The DNA pellet was resuspended in 100 μl TE buffer with 0.01 mg/ml of RNase and a 9 pi aliquot of DNA was run on 0.8% agarose to check for quantity and quality.

### Example 2

DNA Amplification and Clean-Up

Preliminary testing showed that the primer pair, Primer 1 (5′ GGC GTT TGG TGT TAG GTT TGA 3′) (SEQ ID NO:90) and Primer 2 (5′CGT CTG GGA ACA ACC AAA AGT 3′) (SEQ ID NO:91) could amplify in one PCR reaction sufficient amounts of both AHAS1 and AHAS3 sequences for both PM1 and PM2 tests. Each PCR reaction mixture was set up in a total volume of 75 μl containing 1×PCR buffer II (Perkin Elmer), 2.5 mM MgCl₂, 200 μM of each dNTP, 400 nM each of Primer 1 and Primer 2, 100 ng of DNA (or 4 μl of extracted DNA) and 3 units of AmpliTaq® DNA polymerase (Perkin Elmer). Amplification reactions were carried out in Perkin Elmer GeneAmp 9600 or 9700 PCR systems. The PCR program included an initial denaturing step at 94° C., followed by 30 cycles of denaturation at 94° C. for 10 seconds, annealing at 56° C. for 15 seconds, and extension at 72° C. for 30 seconds with a final extension step of 5 minutes at 72° C. An aliquot of the PCR product was checked on 1.4% agarose for an expected product size of 1 Kb.

In the clean-up step, 50 μl of each PCR product was first treated with 10 units of CIP (calf intestinal phosphatase, NEW ENGLAND BioLabs Inc.) by incubating at 37° C. for 1 hour and then deactivating the enzyme by incubating at 72° C. for 15 minutes in Perkin Elmer GeneAmp 9700 PCR systems. Subsequently, the 50 μl aliquot was purified using the QIAquick™ 96 PCR Purification Kit (QIAGEN) and eluted in 50 μl ddH₂O. Samples were then placed in a Universal Vacuum System UVS400/Speed Vac® Plus SC110A (Savant) for approximately 1 hour or until the water in the sample completely evaporated. The CIP treated and purified PCR product was resuspended in ddH₂O at a concentration of approximately 50 ng/μl and was used as DNA templates for the primer extension reactions for detecting the PM1 and PM2 mutations.

### Example 3

Primer Extension PCR for Detecting PM1 and PM2 Mutations Using ABI PRISM®

The ABI PRISM® SNaPshot ddNTP Primer Extension Kit was used on each DNA sample and to detect both the PM1 and the PM2 single nucleotide mutations. The mutation detecting primers are as follows: PM1 extension primer: 5′CAT CTT TGA AAG TGC CAC CA 3′ (SEQ ID NO:24) for detection of the PM1 mutation and PM2 extension primer: 5′ CTT TGT AGA ACC GAT CTT CC 3′ (SEQ ID NO:66) for detection of the PM2 mutation. Primer extension reactions were performed with 100 ng of CIP treated and purified PCR amplified templates in a total volume of 10 μl with 100 nM of the appropriate mutation primer, SNaPshot Ready Reaction Premix as indicated by the manufacturer. Thermal cycling was performed in Perkin Elmer GeneAmp 9600 or 9700 PCR systems with conditions set for 25 cycles of denaturation at 96° C. for 10 seconds, annealing at 50° C. for 5 seconds and extension at 60° C. for 30 seconds. Post-extension treatment consisted of incubating the reaction mixture for 1 hour at 37° C. with 1 unit of calf intestinal phosphatase (NEW ENGLAND BioLabs Inc.) and the enzyme was inactivated at 72° C. for 15 minutes. Samples were then prepared for loading on an ABI PRISM® 3700 DNA Analyzer by adding 1 μl of each post-extension treated reaction to 10 μl of deionized formamide, denatured at 95° C. for 5 minutes and then loaded and run using a GeneScan 5 Run Module. Data was collected and viewed using the ABI PRISM® GeneScan v. 3.5.1 software.

### Example 4

Detection of PM1 and PM2 Mutations in B. napus using ABI PRISM®

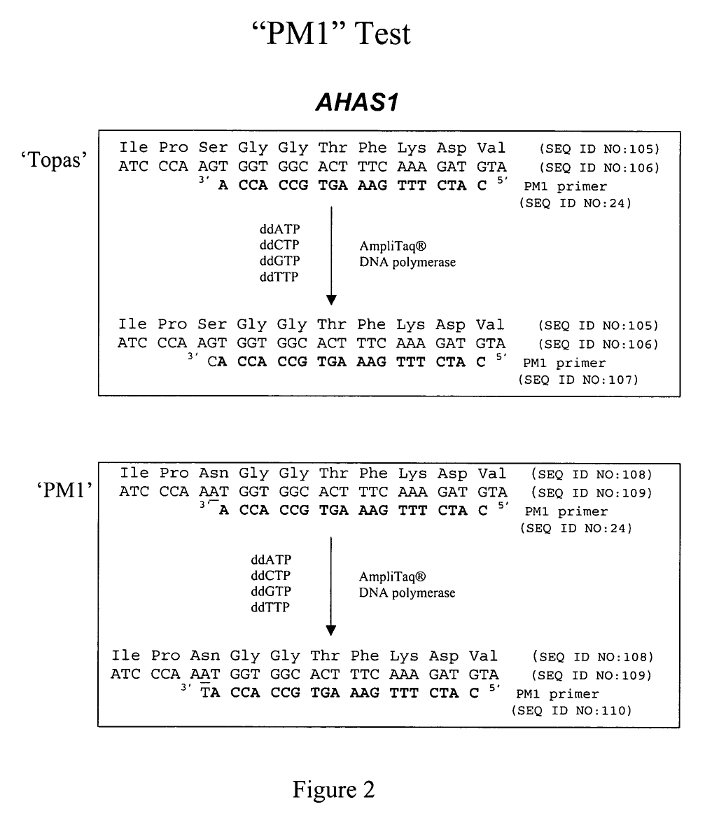

The PM1 test using the primer PM1 involves the extension of the next nucleotide to the primer sequence with the coding strand as the template. Thus, in the wildtype plant, here a B. napus cv. ‘Topas’ plant, the observed nucleotide should be “C” corresponding to the wildtype “G” in the codon “AGT” for Serine on the coding strand. When the test is done on the mutated PM1 B. napus plant, the observed nucleotide should be “T” corresponding to the mutated “A” in the codon “AAT” for Asparagine on the coding strand (FIG. 2). The results obtained with the ABI PRISM® method showed exactly the predicted results. A mutated PM2 B. napus plant that did not contain the PM1 mutation was shown to provide the same results as the wildtype ‘Topas’ plant in the PM1 test. Therefore, the PM1 mutation was detected accurately in B. napus using the ABI PRISM® primer extension methodology.

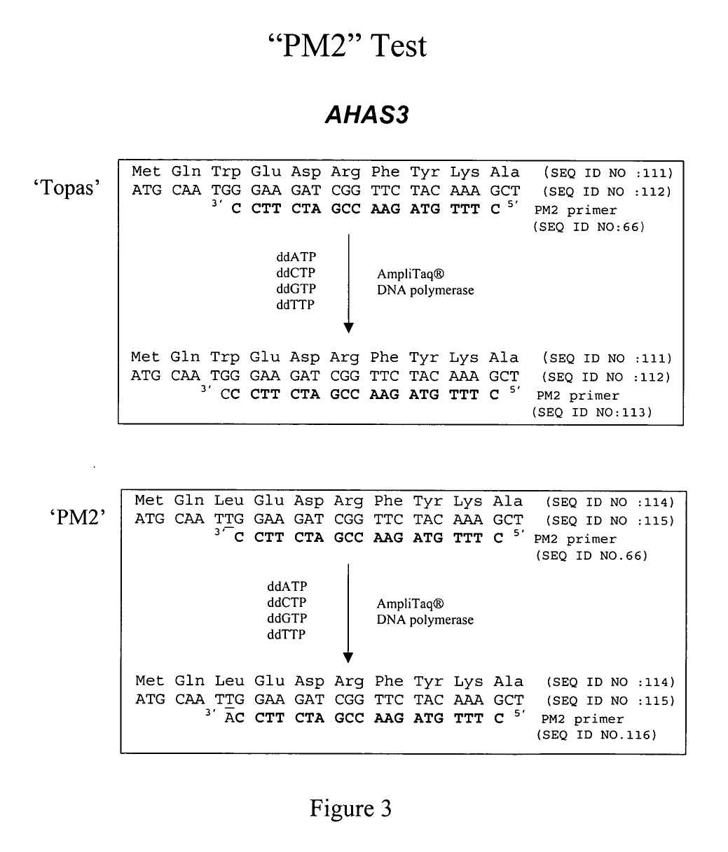

Similarly, the PM2 test using the primer PM2 involves the extension of the next nucleotide to the primer sequence with the coding strand as the template. Thus, in the wildtype plant, e.g. ‘Topas’, the observed nucleotide should be “C” corresponding to the wildtype “G” in the codon “TGG” for Tryptophan on the coding strand. When the test was done on the mutated PM2 B. napus plant, the observed nucleotide should be “A” corresponding to the mutated “T” in the codon “TTG” for Leucine on the coding strand (FIG. 3). The results obtained with the present method showed exactly the predicted results. A mutated PM1 B. napus plant that does not have the PM2 mutation was shown to provide the same results as the wildtype ‘Topas’ plant in the PM2 test. Therefore, the PM2 mutation was detected accurately in B. napus using the ABI PRISM® primer extension methodology.

### Example 5

Validation of ABI PRISM® PM1 and PM2 Detection Method

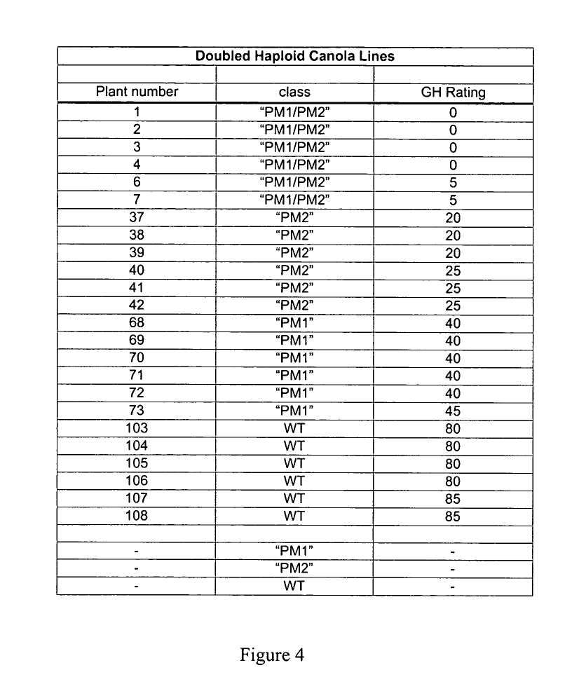

In order to validate the use of the present method on plant materials with a genetic background different from the one used to develop the markers and the method (the B. napus ‘Topas’ plant), the PM1 and PM2 tests were performed to detect the presence or absence of the CLEARFIELD® trait on 24 doubled haploid (DH) (i.e., homozygous) canola lines. These 24 lines were divided into four classes: PM1, PM2, PM1/PM2 and WT based on the results of survival after spraying with herbicide. The codes and classification of the DH lines are summarized in FIG. 4, in which “GH Rating” means greenhouse rating on mortality: 0 means all plants survive after spraying and 85% means 85% of the plants died after spraying. Also included in FIG. 4 are the three controls used in the validation tests: PM1, PM2 and WT, all from the B. napus ‘Topas’ var. used in Examples 2 through 4 for development of the PM1/PM2 assay. The amplification of the templates and the mutation tests were repeated three times for each DH line from Advanta Seeds and twice for the three control samples.

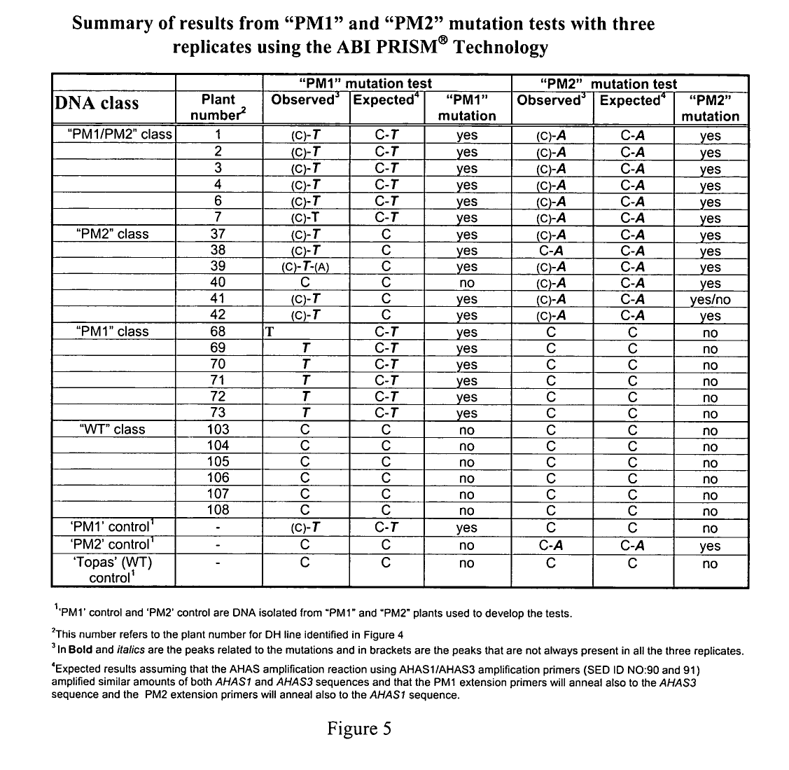

The results of the PM1 and PM2 mutation tests are summarized in FIG. 5. The plant number in FIG. 5 corresponds to the plant number in FIG. 4. Additionally, the peaks related to the mutations are in bold and in italics while the peaks that are not always present or present in various amounts in all the three replicates are in brackets. The “Expected Results” column reflects those results that are expected assuming that the amplification reaction using the primer pair AHAS1/AHAS3 amplification primer of SEQ ID NO: 90 and the AHAS1/AHAS3 amplification primer of SEQ ID NO: 91 amplified similar amounts of both AHAS1 and AHAS3 sequences and that the PM1 extension primers will anneal also to the AHAS3 sequence and the PM2 extension primers will anneal also to the AHAS1 sequence.

As shown in FIG. 5, the observed results for both the PM1 and PM2 mutation tests agreed with the expected results for all six plants in the PM1/PM2 class. With the PM1 class, all six plants showed the PM1 mutation (as “T”). All of the wild-type plants showed the absence of either mutation. Therefore, with all three classes of plants, the present invention can correctly predict the presence or absence of the PM1 and PM2 mutations.

The results for the PM2 class were more complicated. All the six plants of the PM2 classes were expected to have the PM2 mutation (i.e. an “A” with the PM2 mutation test). In fact, all the six plants did detect an “A” with the test throughout the three replicates. The PM2 class was expected to have the wild-type “C” for the PM1 mutation test. However, in the observed results, only plant #40 showed the wild-type “C”, while each of the other five plants consistently showed a “T” for the PM1 mutation test, indicating the unexpected presence of the of the PM1 mutation. The control lines gave the expected results.

It is believed that the discrepancy in the expected and actual results regarding the plants classified as containing only the PM2 mutation is due to misclassification under the herbicide spraying test and that this discrepancy reflects the superiority of the present invention. One advantage of using the present invention to identify the presence or the absence of PM1 or PM2 mutations over the herbicide spraying test is that the present invention can unequivocally tell whether the mutations are present in the genetic materials of the tested plants. Hence, the invention described herein presents a more reliable test which will not be influenced by other environmental factors.

Using the present invention, one can easily tell apart the wild type plants from those with only the PM1 mutation and also differentiate between plants with only PM1 or PM2 mutations and those with both PM1 and PM2 mutations, which are particularly difficult to distinguish using the spraying test. With the prior art herbicide spraying test, a statistical number of plants of the same line need to be grown and sprayed to obtain meaningful results while with the present invention, fewer plants from the same line need to be tested. Since the methods of the present invention only require very small amount of leaf materials per line, another advantage of these methods is that they can be performed when the plants are very young, for example at the cotyledon stage. This advantage translates into savings in growth space and other costs.

### Example 6

Detection of PM1 and PM2 Mutations in B. napus Using PYROSEQUENCING PSQ™ 96

A second method to allow high throughput detection of the presence or absence of “PM1” and “PM2” mutations in B. napus was designed, the method comprising four steps:

1. Isolation of genomic DNA

2. Separation of AHAS1 and AHAS3 DNA template preparations by PCR with an AHAS1-specific forward primer paired with a biotinylated AHAS1/AHAS3 reverse primer for AHAS1 and an AHAS3-specific forward oligonucleotide primer paired with the same biotinylated AHAS1/AHAS3 reverse primer for AHAS3

3. Isolation of single stranded DNA templates

4. PYROSEQUENCING™ reactions with PM1 sequencing primer for detecting the “PM1” mutation and PM2 sequencing primer for detecting the “PM2” mutations.

DNA Isolation

The procedure set forth in Example 1 was used to isolate DNA from plants for analysis using the PYROSEQUENCING™ method.

DNA Amplification

For detection of the PM1 and PM2 mutations using the PYROSEQUENCING™ method, the best results were obtained when AHAS1 and AHAS3 sequences were separately amplified as templates. Therefore, two amplification reactions were first performed using different forward primers, AHAS1-specific forward primer for AHAS1 (5′ GGA AAG CTC GAG GCT TTC GCT 3′; SEQ ID NO:92) and AHAS3-specific forward primer for ALSS3 (5′ GGA AAG CTC GAG GCG TTT GCG 3′; SEQ ID NO: 94) but pairing with the same biotinylated reverse primer, AHAS1/AHAS3 reverse primer (5′ ATC ACC AGC TTC ATC TCT CAG T 3′; SEQ ID NO:93). Each PCR reaction was set up in a total volume of 30 μl containing 1×PCR buffer II (Applied Biosystems, Foster City, Calif.), 2.5 mM MgCl₂, 200 μM of each dNTP, 300 nM each of an AHAS1-specific forward primer and AHAS1/AHAS3 reverse primer for AHAS1 and an AHAS3-specific forward primer and AHAS1/AHAS3 reverse primer for AHAS3, 5 ng of DNA and 1.25 units of AmpliTaq® Gold DNA polymerase (Applied Biosystems, Foster City, Calif.). Amplification reactions were carried out in Applied Biosystems GeneAmp 9600® or GeneAmp 9700® PCR systems. The PCR program includes an initial denaturing step at 94° C. for 10 minutes, followed by 45 cycles of denaturation at 94° C. for 10 seconds, annealing at 56° C. for 15 seconds, and extension at 72° C. for 30 seconds with a final extension step of 10 minutes at 72° C. An aliquot of each PCR product was checked on 1% agarose for an expected product size of 1 Kb.

Single Strand Template Isolation and Annealing of Sequencing Primers for Detection of “PM1” and “PM2” Mutations

PCR amplified products were immobilized by mixing 25 μl of the PCR product with 150 ng of Dynabeads® M-280 Streptavidin (Dynal AS, Oslo, Norway) and 25 μl of 2× Binding-Washing buffer II pH 7.6 (PYROSEQUENCING™) and were incubated on an agitator at 65° for 30 minutes Using the PSQ 96 Sample Prep Tool, the beads carrying the biotinylated templates were then transferred and released into a PSQ 96 Plate containing 50 μl of 0.5 M NaOH per well and left to soak with gentle agitation for 1 minute. The beads now carrying the isolated biotinylated non-coding strands were then transferred into a second PSQ™ 96 Plate for a wash in 100 μl of 1× annealing buffer (PYROSEQUENCING™). Finally, annealing of the sequencing primers was done by transferring the beads into a third PSQ 96 Plate containing 44 μl of 1× annealing buffer (PYROSEQUENCING™) and either 10 pmol of PM1 sequencing primer (5′ GTG TTA CCG ATG ATC C₃′; SEQ ID NO: 95) or 10 pmol of PM2 sequencing primer (5′ GGG ATG GTC ATG CAA T 3′; SEQ ID NO: 96) for assaying the PM1 and PM2 mutations respectively. This third plate was then incubated at 94° C. for 3 minutes and allowed to cool to room temperature for 5 to 10 minutes. SNP detection using the PYROSEQUENCING (PSQ™ 96) system

The third PSQ 96 Plate containing PM1 or PM2 sequencing primers annealed to the non-coding biotinylated strands from each PCR product was loaded onto the PSQ™ 96 system and the pyrosequencing run was carried out using the PSQ™ 96 Instrument Control module from the PSQ™ 96 SNP Software (version 1.2 AQ). The PSQ™ 96 SNP Entry module was used to enter the orders of dispensing nucleotides for both PM1 and PM2 detection (CTAGCTGTG for “PM1” detection and CTGCAGATC for “PM2” detection) while the PSQ™ 96 Evaluation module was used for viewing the results of pyrosequencing.

The choice of the non-coding sequence as the template and the specific sequencing primers combinations for the “PM1” and “PM2” assay was the result of optimization of the process to produce unambiguous pyrograms that could infer the presence or absence of the mutations and whether they are present in the homozygous or heterozygous state.

Results of “PM1” and “PM2” Tests Using Pyrosequencing™

Using the pyrosequencing technology platform for the “PM1” and “PM2” tests requires that the AHAS1 and AHAS3 sequences around the mutations to be amplified separately by specific PCR reactions. In the pyrosequencing technology, the incorporation of each nucleotide with the release of pyrophosphate during the primer extension reaction is coupled to the sulfurylase/luciferase system, which gives light signals proportional to the number of nucleotides incorporated at each elongation step. The results of the pyrosequencing reaction indicate the identity of the nucleotide sequences around the polymorphic site from which the nucleotide at the polymorphic site can be read. With the PM1 test, both B. napus ‘Topas’ and the B. napus ‘PM2’ line have the wildtype AHAS1 sequence and the sequence extended from the PM1 sequencing primer is CAAGTGGTGG (SEQ ID NO:97); while for the mutant PM1 line, the extended sequence is CAAATGGTGG (SEQ ID NO:98) indicating the G→A PM1 mutation on the coding strand. With the PM2 test, both ‘Topas’ and the ‘PM1’ line have wildtype AHAS3 sequence and the sequence extended from the PM2 sequencing primer is GGGAAGATC (SEQ ID NO:99); while for the mutant PM2 line, the extended sequence is TGGAAGATC (SEQ ID NO:100) indicating the G→T PM2 mutation on the coding strand. Thus both PM1 and PM2 mutations were detected accurately using the PYROSEQUENCING™ technology.

Throughout this application, various publications are referenced. The disclosures of all of these publications and those references cited within those publications in are hereby incorporated by reference in their entireties. It should also be understood that the foregoing relates to preferred embodiments of the present invention and that numerous changes may be made therein without departing from the scope of the invention. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

1. A method of assaying a plant for imidazolinone herbicide resistance conferred by the combination of a PM1 mutation of a B. napus AHAS1 gene and a PM2 mutation of a B. napus AHAS3 gene, the method comprising the steps of:
a) isolating genomic DNA from the plant;
b) determining the presence or absence of the PM1 mutation in the DNA; and
c) determining the presence or absence of the PM2 mutation in the DNA, wherein the presence of the PM1 mutation and the PM2 mutation is indicative of commercially relevant imidazolinone tolerance in the plant.

2. The method of claim 1, wherein the plant is a Brassica species.

3. The method of claim 2, wherein the Brassica species is selected from the group consisting of B. napus, B. campestris/rapa, and B. juncea.

4. The method of claim 1, further comprising the step of amplifying the isolated DNA prior to determining the presence or absence of the PM1 and PM2 mutations.

5. The method of claim 1, wherein the determining steps are performed using a primer extension-based single nucleotide polymorphism detection method.

6. A PM1 primer extension oligonucleotide comprising a sequence selected from the group consisting of SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO: 28; SEQ ID NO:29; SEQ ID No;30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42 and SEQ ID NO:95.

7. A PM1 oligonucleotide comprising a sequence selected from the group consisting of SEQ ID NO:45 and SEQ ID NO:46.

8. A PM2 primer extension oligonucleotide comprising a sequence selected from the group consisting of SEQ ID NO:47; SEQ ID NO:48; SEQ ID NO:49; SEQ ID NO:50; SEQ ID NO:51; SEQ ID NO:52; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:69; SEQ ID NO:70; SEQ ID NO:71; SEQ ID NO:72; SEQ ID NO:73; SEQ ID NO:74; SEQ ID NO:75; SEQ ID NO:76; SEQ ID NO:77; SEQ ID NO:78; SEQ ID NO:79; SEQ ID NO:80; SEQ ID NO:81; SEQ ID NO:82; SEQ ID NO:83; SEQ ID NO:84 and SEQ ID NO:96.

9. A PM2 oligonucleotide comprising a sequence selected from the group consisting of SEQ ID NO:87 and SEQ ID NO:88.

10. An oligonucleotide comprising a sequence selected from the group consisting of SEQ ID NO:43; SEQ ID NO:44; SEQ ID NO:85 and SEQ ID NO:86.

11. An amplification oligonucleotide comprising a sequence selected from the group consisting of SEQ ID NO:90; SEQ ID NO:91; SEQ ID NO:92; SEQ ID NO:93; and SEQ ID NO:94.
